(19) United States
(12) Patent Application Publication
(10) Pub. No.: US 2008/0286768 A1
(43) Pub. Date: Nov. 20, 2008

(54) Sequencing a Polymer Molecule

(75) Inventor: Preben Lexow, Oslo (NO)

Correspondence Address: BLANK ROME LLP, 600 NEW HAMPSHIRE AVENUE, N.W., WASHINGTON, DC, 20037, US

(73) Assignee: LINGVITAE AS, Oslo (NO)

(21) Appl. No.: 11/577,033

(22) PCT Filed: Oct. 12, 2005
(86) PCT No.: PCT/GB2005/003926

§ 371 Date: Aug. 13, 2007

(30) Foreign Application Priority Data
Oct. 13, 2004 (GB) 0422733.6

(51) Int. Cl. C12Q 1/68

(52) U.S. Cl. 435/6

(57) ABSTRACT

A method for sequencing a target polymer molecule comprises the steps of: (i) treating the target polymer with an agent that degrades sequentially at least one end of the target polymer; (ii) converting at least a portion of the degraded end of different degraded polymers into a readable signal sequence, and labeling each of said degraded polymers with a tag that represents the relative order of degradation; (iii) determining the sequence of the readable signal sequence; and (iv) determining the sequence of the target polymer using the sequence data obtained in step (iii) and the identification of each associated tag.

### FIELD OF THE INVENTION

This invention relates to methods for sequencing biological polymer molecules. In particular, the method is suitable for sequencing polynucleotides.

### BACKGROUND OF THE INVENTION

Advances in the study of molecules have been led, in part, by improvement in technologies used to, characterise the molecules or their biological reactions. In particular, the study of the nucleic acids DNA and RNA has benefited from developing technologies used for sequence analysis and the study of hybridisation events.

The principal method in general use for large-scale DNA sequencing is the chain termination method. This method was first developed by Sanger and Coulson (Sanger et al., Proc. Natl. Acad. Sci. USA, 1977; 74: 5463-5467), and relies on the use of dideoxy derivatives of the four nucleotides which are incorporated into the nascent polynucleotide chain in a polymerase reaction. Upon incorporation, the dideoxy derivatives terminate the polymerase reaction and the products are then separated by gel electrophoresis and analysed to reveal the position at which the particular dideoxy. derivative was incorporated into the chain.

Although this method is widely used and produces reliable results, it is recognised that it is slow, labour-intensive and expensive.

U.S. Pat. No. 5,302,509 discloses a method to sequence a polynucleotide immobilised on a solid support. The method relies on the incorporation of 3′-blocked bases A, G, C and T having a different fluorescent label to the immobilised polynucleotide, in the presence of DNA polymerase. The polymerase incorporates a base complementary to the target polynucleotide, but is prevented from further addition by the 3′-blocking group. The label of the incorporated base can then be determined and the blocking group removed by chemical cleavage to allow further polymerisation to occur. However, the need to remove the blocking groups in this manner is time-consuming and must be performed with high efficiency.

WO-A-00/39333 describes a method for sequencing a polynucleotide by converting the sequence of a target polynucleotide into a second polynucleotide having a defined sequence and positional information contained therein. The sequence information of the target is said to be “magnified” in the second polynucleotide, allowing greater ease of distinguishing between the individual bases on the target molecule. This is achieved using “magnifying tags” which are predetermined nucleic acid sequences. Each of the bases adenine, cytosine, guanine and thymine on the target molecule is represented by an individual magnifying tag, converting the original target sequence into a magnified sequence. Conventional techniques may then be used to determine the order of the magnifying tags, and thereby determining the specific sequence on the target polynucleotide.

Although useful, sequencing long polymers is still problematic and requires the sequencing of a large number of polymer fragments followed by substantial sequence reconstruction. There is a constant need to increase read lengths and simplify the reconstruction required, particularly when sequencing a polymer de novo.

### SUMMARY OF THE INVENTION

The present invention is based on the realisation that a target polymer can be sequenced by encoding positional and sequence information into fragments produced by sequential degradation of the target polymer. These fragments can be used to reconstruct the sequence of the target polymer.

According to a first aspect of the invention, a method for sequencing a target polymer molecule comprises the steps of:

(i) treating the target polymer with an agent that degrades sequentially at least one end of the target polymer;

(ii) converting at least a portion of the degraded end of different degraded polymers into a readable signal sequence, and labelling each of said degraded polymers with a tag that represents the relative order of degradation;

(iii) determining the sequence of the readable signal sequence; and

(iv) determining the sequence of the target polymer using the sequence data obtained in step (iii) and the identification of each associated tag.

### DETAILED DESCRIPTION OF THE INVENTION

The present invention is used to determine the sequence of a target polymer molecule. The method is particularly useful for de novo sequencing.

The method of the invention has the following general steps: firstly, a target polymer is sequentially degraded. Each fragment is then labelled with two labels. A first label, referred to as a “readable signal sequence” contains information on the sequence of the fragment. A second label, referred to as a “positional tag”, is added to indicate the point at which the fragment was removed from the degradation reaction. Once all the fragments have been labelled with a “readable signal sequence” and a “positional tag”, these labels are detected, providing information on the sequence of each fragment and its position in the target polynucleotide. This information can then be used to determine the sequence of the target polymer, by collating the type and order of each sequenced fragment.

Preferably, the degradation reaction is followed by removal of samples and placing the samples in discrete compartments for analysis. Each sample therefore contains a fragment of the target polymer that is a different length, and therefore has a different sequence at the degraded end in comparison to the other fragments.

The method provides sequence information on a target polymer. As used herein, the term “polymer” refers to any molecule comprised of linked monomer units. Preferably, the polymer is a biological polymer, in particular a polynucleotide or polypeptide. The term “polynucleotide” is well-known in the art and is used to refer to a series of linked nucleic acid bases, e.g. DNA or RNA. Nucleic acid mimics, including PNA (peptide nucleic acid), LNA (locked nucleic acid) and 2-O-methRNA are also within the scope of the invention. The target polynucleotide may be single-stranded or double-stranded.

As used herein, the term “base” refers to each nucleic acid monomer, A, T(U), G or C. These abbreviations represent the nucleotide bases adenine, thymine (uracil), guanine and cytosine. Uracil replaces thymine when the polynucleotide is RNA, or it can be introduced into DNA using dUTP, again as well understood in the art.

The term “polypeptide” is also well-known in the art, and is used to refer to a series of linked amino acid molecules. The term is intended to include both short peptide sequences and longer protein sequences.

The method of the invention involves the sequential degradation of the target polymer, to create fragments of varying length. Degradation may occur from one end, or both ends, of the target polymer. Methods for sequentially-degrading target polymers are well-known in the art, for example enzymatic digestion. It will be appreciated by one skilled in the art that nucleases are suitable for the degradation of a polynucleotide, and proteases and peptidases are suitable for the degradation of polypeptides. In a preferred embodiment, an exonuclease or exoprotease is used, under conditions suitable for enzyme activity; these enzymes sequentially remove the terminal monomer units from respectively, a polynucleotide and a polypeptide. Conditions suitable for enzyme activity will be apparent to one skilled in the art.

During the sequential degradation reaction, samples of degraded target polymer are preferably removed from the reaction mix at specific time intervals and placed into discrete compartments. Each discrete compartment will therefore contain a fragment of different length; a fragment removed early in the degradation reaction will be a longer fragment than one removed late in the degradation reaction. A sample may also be removed prior to initiating the degradation reaction, this first sample will therefore contain the full length target polymer. Any number of samples may be removed during the degradation reaction, preferably at pre-determined time intervals, designed to optimise the number of fragments generated. As used herein, the term “sample fragment” refers to the fragments that are removed during degradation.

On removal from the reaction mix, it will be necessary stop the degradation reaction. Methods suitable for stopping an enzymatic reaction will be apparent to one skilled in the art. Changes in temperature and pH are known to inactivate enzymes, as is the addition of an inhibitor. Preferably, the technique used to stop degradation does not damage or adversely effect the sample fragments. If an exonuclease is used to fragment the sample, the exonuclease may be inactivated by techniques known in the art. For example, addition of a buffer containing Tris base and EDTA followed by heating to 70° C. inactivates exonuclease III. This technique is used in the Erase-a-Base technique (Promega Corporation), where 1 μl of S1 nuclease stop buffer (0.3M Tris base, 0.05M EDTA) is added to a 2.5 μl reaction volume and heated to 70° C. for 10 minutes (see Promega Erase-a-Base system technical manual #006, available from www.promega.com and also Henikoff, Nucleic Acids Res. 1990 May 25; 18(10): 2961-2966).

An alternative technique that can be used to stop the degradation reaction is to remove the degradation enzyme from the sample. Techniques suitable for the specific removal of an enzyme from a mixture are well known in the art, for example the use of affinity chromatography, wherein a binding partner of the enzyme is immobilised and the enzyme is removed from the sample as it contacts the immobilised affinity partner. Alternatively, each target polymer may be immobilised to a solid support prior to the degradation reaction; preferably the target polymer is immobilised onto beads that allow aliquots to be removed during the degradation reaction. Each sample of beads that is removed during the degradation reaction will have the sample fragments immobilised thereon. These sampled beads can then be washed to remove the enzyme, as will be appreciated by one skilled in the art. In this embodiment, it is desirable to ensure that the beads with the polymers attached maintain a homogenous mixture during the degradation reaction to ensure uniform degradation. This can be achieved by simple agitation or stirring of the beads.

Methods of immobilising biological polymers onto a support material, such as beads, are well known in the art, for example polynucleotides may be immobilised by the use of biotin-avidin interactions, photolithographic techniques and techniques that rely on “spotting” individual polymers in defined positions on a support material.

Immobilisation may be by specific covalent or non-covalent interactions. The interaction should be sufficient to maintain the polymers on the support during washing steps to remove unwanted reaction components. Immobilisation will preferably be at one end only, e.g either the 5′ or 3′ terminus of a polynucleotide, so that the polymer is attached to the support at the end only. However, the polymer may be attached to the support at any position along its length, the attachment acting to tether the polynucleotide to the support.

The skilled person will appreciate the appropriate means to immobilise the polymer to the support material. Suitable coatings may be applied to the support to facilitate immobilisation, as will be appreciated by the skilled person. Suitable coatings for attaching polynucleotides include epoxy coatings (e.g. 3-glycidyloxypropyltrimethoxysilane), superaldehyde coating, mercaptosilane, and isothiocyanate. Alternatively, several linker groups may be used, including PAMAM dendritic structures (Benters et al., Chem Biochem., 2001; 2: 686-694) and the immobilisation linkers described in Zhao et al., Nucleic Acids Research, 2001; 29(4): 955-959.

In an alternative embodiment, the degradation reaction is not stopped immediately. Instead, the readable signal sequence may be attached to the sample fragment immediately after removal from the degradation reaction.

At least a portion of each sample fragment is converted into a readable signal sequence. Any portion may be converted, between a single base and the entire sample fragment. Preferably, at least three monomer units from each sample fragment are converted, more preferably between 3 and 100 monomers, e.g. 20 monomer units. If the target polymer is degraded from one end only, at least the corresponding end of each sample fragment is converted into a readable signal sequence. For example, if degradation occurs from the 3′ end of a target polynucleotide, at least the three 3′ bases in the sample fragment are converted into a readable signal sequence. If both ends of the target are degraded, either end, or both ends, of each fragment can be converted. In a preferred embodiment, the entire sequence of each sample fragment is converted into a readable signal sequence. Most preferably, the combined readable signal sequences of all of the sample fragments represent the entire sequence of the target polynucleotide.

As used herein, the term “readable signal sequence” refers to a sequence that comprises a label, or the means for attaching a label, that enables at least a portion of the sequence to be identified in a subsequent read-out step. Any label may be used; methods of sequencing biological polymers using a label are well known in the art. For example, a polypeptide can be converted into a readable signal sequence by the addition of a reagent that reacts with the N-terminal amino acid residue and allows the identification of the terminal residue in a subsequent read-out step. Commonly used reagents include dansyl chloride and phenylisothiocyanate (PITC). PITC is used in the “Edman Degradation” method of polypeptide sequencing, which is well known in the art. A polynucleotide can be converted into a readable signal sequence using any suitable technique. The chain-termination (“Sanger”) method of polynucleotide sequencing can be used, wherein the sample fragment is converted into a readable signal sequence that contains a dideoxynucleoside triphosphate.

It will be appreciated by one skilled in the art that in order to obtain the sequence of a series of monomer units in the sample fragment, a number of sequencing cycles may be required. This is within the scope of the present invention.

In a preferred embodiment, the readable signal sequence is a polynucleotide which comprises at least two bases representing a single monomer unit in the sample fragment. The sequence information of the sample fragment is said to be “magnified” in the readable signal sequence, allowing greater ease of distinguishing between the individual bases on the target molecule. These preferred readable signal sequences which have previously been described as “magnified (or “magnifying”) tag” sequences, are referred to herein as “magnified readable signal sequences”. Examples of these sequences are given in WO-A-00/39333 and WO04/94663, which are both incorporated herein by reference. Any biological polymer may be converted into a magnified readable signal sequence, as is known in the prior art. WO-A-00/39333 describes the conversion of a polynucleotide into a magnified readable signal sequence. The conversion of proteins and peptides into polynucleotide magnified readable signal sequences is described in WO04/94663, which is incorporated herein by reference.

Each magnified readable signal sequence will preferably comprise two or more nucleotide bases, preferably from 2 to 50 bases, more preferably 2 to 20 bases and most preferably 4 to 10 bases, e.g. 6 bases. In a preferred embodiment, there are three different bases in each magnified readable signal sequence. For example, one base will be complementary to a labelled nucleotide introduced during the read-out step, one base will act as a “spacer” to provide separation between incorporated labels, and one base will act as a stop signal.

A binary code may be included in the magnified readable signal sequence, as disclosed in co-pending application number PCT/GB04/01665. In this “binary” embodiment, each magnified readable signal sequence comprises two units of distinct sequence which represent all of the four bases on the sample fragment. The two units are used as a binary system, with one unit representing “0” and the other representing “1”. Each base on the sample fragment is characterised by a combination of the two units in the magnified readable signal sequence. For example, adenine may be represented by “0”+“0”, cytosine by “0”+“1”, guanine by “1”+“0” and thymine by “1”+“1”. It is necessary to distinguish between the units, and so a “stop” signal can be incorporated into each unit. It is also preferable to use different units representing “1” and “0”, depending on whether the base on the sample fragment is in an odd or even numbered position.

This is demonstrated as follows:

Odd numbered template sequence:
“0”: TTTTTTA(CCC)
“1”: TTTTTTG(CCC)
Even numbered template sequence:
“0”: CCCCCCA(TTT)
“1”: CCCCCCG(TTT)

In this example, the underlined base is the target for labelled nucleotides in a polymerase reaction, the bases in parentheses are used as a stop signal, and the remaining bases are to provide separation between the labels.

It is preferred that a plurality of monomer units in the sample fragment are converted into magnified readable signal sequences. Each magnified readable signal sequence remains attached to the target polymer in series, thereby forming a single polynucleotide molecule containing a series of magnified readable signal sequence units, that encodes the sequence of the target polymer.

It is possible to distinguish the different magnified readable signal sequences during a “read-out” step, e.g. involving either the incorporation of detectably labelled nucleotides in a polymerisation reaction, or on hybridisation of complementary oligonucleotides, or in a conventional sequencing reaction. In the above example, incorporation of detectably labelled nucleotides may be used. In odd numbered positions (1, 3, 5, etc) the nucleotide mix, introduced during the polymerase reaction, consists of Fluor X-dUTP, Fluor Y-dCTP and dATP (dGTP is missing from the mix). The complementary base for Fluor Y is missing for “0”, and the complementary base for Fluor X is missing for “1”. Accordingly, during a polymerase reaction, if the unit “0” is present, it will be possible to detect this by monitoring for Fluor X, and if “1” is present, by monitoring for Fluor Y.

In all even numbered positions (2, 4, 6, etc) the nucleotide mix consists of the same two fluor-labelled nucleotides, but dGTP is used, not dATP, and one or more T bases define the stop signal.

After each magnified readable signal sequence has been “read” it is possible to restart the process by introducing the missing complementary nucleotide (e.g. either dGTP or dATP) to allow incorporation at the stop sequence. Non-incorporated nucleotides are washed away prior to the next read-out step.

Each sample fragment may be converted into the magnified readable signal sequence (or series thereof) using methods known in the art. The conversion method disclosed in WO-A-00/39333, using restriction enzymes, may be adopted. For example, if the sample fragment is a polynucleotide, the sample fragment may be ligated into a vector which carries a class IIS restriction site close to the point of insertion, or the sample fragment may be engineered to contain such a site. The appropriate class IIS restriction enzyme is then used to cleave the restriction site, resulting in an overhang in the sample fragment.

Appropriate adapters which contain one or more of the magnified readable signal sequences units may then be used to bind to one or more of the bases of the overhang. Once the overhang of the adapter and the cleaved vector have been hybridised, these molecules may be ligated. This will only be achieved where full complementarity along the full extent of the overhang is achieved. Blunt-end ligation may then be effected to join the other end of the adapter to the vector. By appropriate placement of a further class II restriction site (or other appropriate restriction enzyme site), which may be same or different to the previously used enzyme, cleavage may be effected such that an overhang is created in the target sequence downstream of the sequence to which the first adapter was directed. In this way, adjacent or overlapping sequences may be consecutively converted into sequences carrying the units of defined sequence.

After conversion into a readable signal sequence but before the read-out step, the sample fragment in each discrete compartment may optionally be immobilised onto a solid support, for example to form an array. Methods of immobilising biological polymers to a support material are well known in the art, as described above. Immobilisation may be carried out by the random distribution of polynucleotides on microbeads, nanoparticles and planar surfaces. Suitable support materials are known in the art, and include glass slides, ceramic and silicon surfaces and plastics materials. The support is usually a flat (planar) surface.

The sample fragment may be immobilised on the support material to form arrays which may form a random or ordered pattern on the solid support. Preferably, the arrays that are used are single molecule arrays that comprise sample fragments in distinct optically resolvable areas, e.g. polynucleotide arrays are disclosed in WO-A-00/06770, the content of which is incorporated herein by reference.

Preferably, each sample fragment contains a readable signal sequence that is complementary to a readable signal sequence of at least one other sample fragment. More preferably, the complementarity is between a plurality of readable signal sequences that represent a plurality of monomer units on a sample fragment, for example between 2 and 20 bases, such as 3, 4 or 5 bases in a polynucleotide. This ensures that there is an overlap between the readable signal sequence information in separate sample fragments, allowing the target sequence to be reconstructed based upon these redundant overlap regions, as will be appreciated by one skilled in the art. The greater the complementarity between readable signal sequences on different sample fragments, the simpler the sequence reconstruction will be.

In addition to at least a portion of each sample fragment being labelled with a readable signal sequence, each fragment is also labelled with a “positional tag” that represents the time at which the fragment was removed from the degradation reaction. In a preferred embodiment, each sample fragment is labelled with a different positional tag, thereby identifying the point at which it was removed from the degradation reaction. Any tag suitable for labelling biological polymers may be used. In a preferred embodiment, the positional tag is a fluorophore. Suitable fluorophores are well known in the art, for example:

Alexa dyes (Molecular Probes)
BODIPY dyes (Molecular Probes)
Cyanine dyes (Amersham Biosciences Ltd.)

### Tetramethylrhodamine (Perkin Elmer, Molecular Probes, Roche Diagnostics)

### Coumarin (Perkin Elmer)

### Texas Red (Molecular Probes)

### Fluorescein (Perkin Elmer, Molecular Probes, Roche Diagnostics)

Any fluorescent detection technique may be used to detect the fluorophore in the read-out step, as will be apparent to the skilled person. Examples of fluorophore detection techniques are outlined below.

In an alternative preferred embodiment, the positional tag is a “magnified tag” of pre-determined sequence. For the avoidance of doubt, a magnified tag comprises two or more bases, as described above and in WO-A-00/39333. Preferably, the positional tag is a polynucleotide comprising a pre-determined series of magnifying tags. When the magnified tag is used as a positional tag, it does not represent the sequence of the sample fragment; it is a pre-determined sequence that is recognisable in a read-out step. By having the readable signal sequence and positional tag in the form of polynucleotides comprising distinct units of two or more bases, i.e. “magnified tags”, the read-out step is simplified, as both the readable signal sequence and positional tag can be read using the same technique. Any method of attaching the magnified tag to the sample fragment may be used. Preferably, the restriction enzyme/ligation based technique disclosed in WO-A-00/39333 (and summarised herein) is used.

The positional tag may be attached directly to the sample fragment, or may be attached to the readable signal sequence. In a preferred embodiment, when both the readable signal sequence and positional tag are magnified tags comprising distinct units of two or more bases, the positional tag and readable signal sequence are continuous, forming a single polynucleotide chain containing both labels. Alternatively, the positional tag and readable signal sequence are linked to opposite terminii of the sample fragment.

Once at least a portion of each sample fragment has been labelled with a readable signal sequence that encodes the sequence of the sample fragment, and a positional tag that indicates the position in the degradation reaction, the data contained within each fragment is detected in a read-out step, thereby identifying the sequence of each fragment and its position in the target molecule. These sequenced fragments can then be reassembled to give the sequence of the target polymer. When the tag and readable signal sequence are both magnified tag sequences, the read-out step may be performed using any suitable technique, for example as described in WO-A-00/39333 and PCT/GB04/01665 and summarised herein. A preferred detection technique is as discussed above, using the polymerase reaction to incorporate bases complementary to those on the readable signal sequence, using either selected, detectably-labelled nucleotides or nucleotides that incorporate a group for subsequent indirect labelling, and monitoring any incorporation event.

To carry out the polymerase reaction-based read-out step it will usually be necessary to first anneal a primer sequence to the magnified readable signal sequence polynucleotide, the primer sequence being recognised by the polymerase enzyme and acting as an initiation site for the subsequent extension of the complementary strand. The primer sequence may be added as a separate component with respect to the polynucleotide, which comprises a complementary sequence that allows the primer to anneal. The polymerase reaction is preferably carried out under conditions that permit the controlled incorporation of complementary nucleotides one unit at a time. This enables each magnified signal sequence unit to be categorised by the detection of an incorporated label. As each unit preferably comprises a “stop” sequence, it is possible to control incorporation by supplying only those nucleotides required for incorporation onto the first unit, as described above. As each unit is recognised by a specific label, it is possible to distinguish between two different units (0 and 1) within each cycle. This enables detection of any incorporated label, and allows the identification and position of the unit to be determined.

When both the readable signal sequence and positional tag are magnified tag sequences, the read-out method may be carried out as follows:
(i) contacting the readable signal sequence comprising the defined units with at least one of the nucleotides dATP, dTTP, dGTP and dCTP, under conditions that permit the polymerisation reaction to proceed, wherein the at least one nucleotide comprises a detectable label specific for that nucleotide;
(ii) removing any non-incorporated nucleotides and detecting any incorporation events;
(iii) removing the label from any incorporated nucleotide; and
(iv) repeating steps ii) to iv), to thereby identify the different units, and thereby the sequence of the target polynucleotide.

The number of different nucleotides required in step (i) of each cycle will be dependent on the design of the magnified signal sequence units. If each unit comprises only one base type, then only one nucleotide (detectably labelled) is required. However, if two bases are utilised (one as a target for the detectably labelled nucleotide and one to provide a gap between different target bases) then two nucleotides will be required (one to bind to the target base and one to “fill in” the bases between the target bases).

The use of a base as a stop signal allows the detection steps to be performed without the requirement for blocked nucleotide's to prevent uncontrolled incorporation during the polymerase reaction. The stop signal is effective as the complement for the “stop” base is absent from the polymerase mix. Therefore, each unit can be characterised before a “fill-in” step is performed, using the missing nucleotide, to incorporate a complement to the stop base, which allows the next unit to be characterised. This is carried out after the detection step. The “stop” base of one unit will not be of the same type as the first base of the subsequent unit. This ensures that the “fill-in” procedure does not progress to the next unit. Non-incorporated nucleotides used in the “fill-in” procedure can then be removed, and the next unit can then be characterised.

The choice of polymerase and detectable label will be apparent to the skilled person. The following is used as a guide only:

a) Klenow and Klenow (exo-) can efficiently incorporate Tetramethylrhodamine-4-dUTP and Rhodamin-110-dCTP (Amersham Pharmacia Biotech) (Brakmann and Nieckchen, 2001, Brakmann and Löbermann, 2000).
b) Vent, Taq and Tgo DNA polymerase can efficiently incorporate dioxigenin and fluorophores like AMCA, Tetramethylrhodamin, fluorescein and Cy5 without spacing at least up to a few positions (Augustin et al., (provide reference?) 2001).
c) T4 DNA polymerase is efficient in filling-in fluorophore labelled nucleotides.

The preferred polymerases are Klenow Large fragment (exo-) and T4 DNA polymerase.

Other conditions necessary for carrying out the polymerase reaction, including temperature, pH, buffer compositions etc., will be apparent to those skilled in the art. The polymerisation step is likely to proceed for a time sufficient to allow incorporation of bases to the first unit. Non-incorporated nucleotides are then removed, for example, by subjecting the array to a washing step, and detection of the incorporated labels may then be carried out.

An alternative read-out strategy is to use short detectably labelled oligonucleotides to hybridise to the units on the magnified readable signal sequence and/or positional tag, and to detect any hybridisation event. The short oligonucleotides have a sequence complementary to specific units of the readable signal sequence. For example, if a binary system is used and each monomer in the sample fragment is defined by a different combination of magnified readable signal sequence units (one representing “0” and one representing “1”) the invention will require an oligonucleotide specific for the “1” unit. In this embodiment, selective hybridisation of oligonucleotides can be achieved by designing each unit to be of a different polynucleotide sequence with respect to other units. This ensures that a hybridisation event will only occur if the specific unit is present, and the detection of hybridisation events identifies the characteristics on the sample fragment.

In a preferred embodiment, the label is a fluorescent moiety. Many examples of fluorophores that may be used are known in the prior art, as indicated above. The attachment of a suitable fluorophore to a nucleotide can be carried out by conventional means. Suitably labelled nucleotides are also available from commercial sources. The label is attached in a way that permits removal, after the detection step. This may be carried out by any conventional method, including:

I. Attacking the signal itself:

### d) Bleaching

i) Photobleaching
ii) Chemical bleaching
   a) Quenching of fluorescence
i) By antibodies raised against the fluor (e.g. anti-fluorescein, anti-Oregon green)
ii) By FRET (the incorporation of a quencher next to a signal can be used to quench the signal, e.g. Taqman-strategy)
   b) Cleavage of signal
i) Chemical cleavage (e.g. reduction of a disulfide bridge between the base and the signal)
ii) Photocleavage (e.g. introduction of a nitrobenzyl ortertbutylketon group)
iii) Enzymatic (e.g. α-chymotryspin digestion of peptide linker).
   II. The signal bearing nucleotide:
   b) Exonucleolytic removal
i) 3′-5′ Exonucleolytic degradation of filled-in nucleotides (e.g. exonuclease III or by activating the 3′-5′ exonucleolytic activity of DNA polymerase when there is an absence of certain nucleotides)
   c) Restriction enzyme digestion
ii) Digestion of double-stranded DNA bearing the signal (e.g. ApaI, DraI, SmaI sites which can be incorporated at the stop signals).

An alternative to the use of labels that permit removal, is to use inactivated labels that are reactivated during a biochemical process.

The preferred method is by photo or chemical cleavage.

When the label is a fluorophore, the fluorescent signal generated on incorporation may be measured by optical means, e.g. by a confocal microscope. Alternatively, a sensitive 2-D detector, such as a charge-coupled detector (CCD), can be used to visualise the individual signals generated.

The general set-up for optical detection is as follows:

| | |
|---|---|
| Microscope: | Epi-fluorescence |
| Objective: | Oil emersion (100X, 1.3 NA) |
| Light source: | Lasers or lamp |
| Filters: | Bandpass |
| Mirrors: | Dichroic mirror and dichroic wedge |
| Detectors: | Photomultiplier tubes (PMT) or CCD camera |

Variants may also be used, including:

A. Total Internal Reflection Fluorescence Microscopy (TIRFM)

| | |
|---|---|
| Light source: | One or more lasers |
| Background | No pinhole required |
| control: | |
| Detection: | CCD camera (video and digital imaging systems) |

B. Confocal Laser Scanning Microscopy (CLSM)

| | |
|---|---|
| Light source: | One or more lasers |
| Background | One or several pinhole apertures |
| reduction: | |
| Detection: | a) A single pinhole: Photomultiplier tube (PMT) |
| | detectors for different fluorescent wavelengths |
| | [The final image is built up point by point |
| | and over time by a computer]. |
| | b) Several thousands pinholes (spinning Nipkow |
| | disk): CCD camera detection of image [The final |
| | image can be directly recorded by the camera] |

C. Two-Photon (TPLSM) and Multiphoton Laser Scanning Microscopy

| | |
|---|---|
| Light source: | One or more lasers |
| Background | No pinhole required |
| control: | |
| Detection: | CCD camera (video and digital imaging systems) |

The preferred methods are TIRFM and confocal microscopy.

It will be appreciated that although specific examples of techniques suitable for magnified readable signal sequence are given herein, the magnified readable signal sequences and “magnified tag” positional tags may be read using any suitable read-out platform.

When the readable signal sequence is not a magnified readable signal sequence, for example it is a PITC-labelled polypeptide or a ddNTP-labelled polynucleotide, any suitable read-out step can be used. Chromatographic and electrophoretic read-out steps are commonly used, as is well-known in the art.

Once the sequence of each fragment is known, it will be apparent to the skilled person that the sequence of the target polymer molecule can be reconstructed, based upon the positional tags that indicate the order of each fragment within the target molecule. The overlapping regions in each readable signal sequence may also aid sequence reinstruction. This may be achieved using conventional software programmes. The content of each of the publications referred to herein are hereby incorporated.

1. A method for sequencing a target polymer molecule, comprising the steps of:
(i) treating the target polymer with an agent that degrades sequentially at least one end of the target polymer;
(ii) converting at least a portion of the degraded end of different degraded polymers into a readable signal sequence, and labeling each of said degraded polymers with a tag that represents the relative order of degradation;
(iii) determining the sequence of the readable signal sequence; and
(iv) determining the sequence of the target polymer using the sequence data obtained in step (iii) and the identification of each associated tag.

2. The method according to claim 1, wherein samples of degraded polymer are removed at pre-determined time points during the degradation reaction and placed into separate compartments for analysis.

3. The method according to claim 1, wherein each readable signal sequence contains a region complementary to a readable signal sequence of at least one other degraded polymer.

4. The method according to claim 1, wherein the combined readable signal sequences of all degraded polymers represents the sequence
of the target polymer.

5. The method according to claim 1, wherein the target polymer is a polynucleotide.

6. The method according to claim 5, wherein the polynucleotide is DNA.

7. The method according to claim 1, wherein the target polymer is a polypeptide.

8. The method according to claim 1, wherein the agent is an exonuclease.

9. The method according to claim 7, wherein the agent is a protease.

10. The method according to claim 1, wherein the readable signal sequence is or comprises a magnifying tag.

11. The method according to claim 1, wherein the tag is or comprises a magnifying tag of predetermined sequence.

12. The method according to claim 1, wherein the tag is a fluorophore.
